(19) United States
(12) Patent Application Publication
(10) Pub. No.: US 2025/0384969 A1
(43) Pub. Date: Dec. 18, 2025

(54) METHOD FOR ACQUIRING CHARACTERISTIC PEAKS OF COMPOUNDS, METHOD FOR CALCULATING THE SIGNAL-TO-NOISE RATIO OF CHARACTERISTIC PEAKS, AND A SERVER FOR EXECUTING THE METHODS THEREOF

(71) Applicant: Agricultural Chemicals Research Institute, Ministry of Agriculture, Taichung City (TW)

(72) Inventors: Ling-Yang WANG, Taichung City (TW); Shao-Kai LIN, Taichung City (TW); Wei-Chen CHUANG, Taichung City (TW)

(21) Appl. No.: 18/934,620

(22) Filed: Nov. 1, 2024

(30) Foreign Application Priority Data
Jun. 14, 2024 (TW) 113122150

(51) Int. Cl. G16C 20/20

(52) U.S. Cl. CPC G16C 20/20

(57) ABSTRACT

A method for obtaining characteristic peaks of compounds, wherein one or more characteristic peaks of compounds are obtained from a mass spectrometry file based on a parameter table. The parameter table records a filter parameter set for each compound, and the selection of characteristic peaks for each compound is determined by the filtering rules defined by its own filter parameter set. Another method for obtaining characteristic peaks of compounds is based on the positions of the characteristic peaks obtained from the mass spectrometry file, from which one or more characteristic peaks of compounds are obtained from another mass spectrometry file.

### FIELD OF INVENTION

The present invention relates to mass spectrometry data analysis, and more particularly to a method for obtaining characteristic peaks of compounds.

### RELATED PRIOR ART

The compounds present in a sample, such as the pesticide residues in a vegetable sample, can be detected using a mass spectrometer. A common mass spectrometer includes a Liquid Chromatography Tandem Mass Spectrometer (LC-MS/MS) and a Gas Chromatography Tandem Mass Spectrometer (GC-MS/MS).

A bottleneck in current compound detection operations is that the mass spectrometry file produced by the mass spectrometer requires experienced examiners to spend a significant amount of manpower and time analyzing it in order to obtain the characteristic peak data of each compound in the file (such as the total area and signal-to-noise ratio of the quantitative or qualitative characteristic peak for each compound). This not only results in inefficient overall compound detection operations but also increases the likelihood of errors due to human oversight. Additionally, it takes 2 to 3 years to train examiners capable of performing such operations independently, leading to frequent shortages of qualified examiners.

### SUMMARY OF THE INVENTION

The present invention provides a method for obtaining characteristic peaks of a compound, comprising: reading mass spectrometry data from a mass spectrometry file, wherein the mass spectrometry data includes a plurality of ion pairs belonging to the compound and all their peaks; reading a filter parameter set for the compound from a parameter table; selecting an ion pair with the highest or lowest peak from the ion pairs of the compound as the quantitative ion pair based on a select parameter from the filter parameter set; and selecting the top N ranked peaks in height from all the peaks of the quantitative ion pair as the quantitative characteristic peaks of the compound based on a quantity parameter from the filter parameter set, where the value of N is determined by the quantity parameter.

In one embodiment, the method of the present invention includes: prior to reading the parameter table, obtaining the name of the compound by querying a compound list based on an ion pair from the mass spectrometry data, wherein the compound list records the names and ion pairs of each compound intended to be detected by the mass spectrometer, and the parameter table records the names of each compound in the compound list and the filter parameter set.

In one embodiment, the parameter table of the present invention is established for the mass spectrometer that generated the mass spectrometry file.

In one embodiment, the method of the present invention includes: based on the position of the quantitative characteristic peaks of the compound, identifying peaks with the same or similar positions from all peaks of ion pairs other than the quantitative ion pair as the quantitative characteristic peaks of the compound.

In one embodiment, the method of the present invention includes: defining an allowable position range for each of the identified quantitative characteristic peaks based on a position parameter from the filter parameter set; and selecting peaks from all the peaks of ion pairs other than the quantitative ion pair, where the positions of the peaks fall within the allowable position range, as the quantitative characteristic peaks of the compound.

In one embodiment, the method of the present invention includes: after obtaining the qualitative characteristic peaks of the compound, if the quantitative ion pair is selected from the ion pair with the lowest peak, a swapping operation will be conducted to interchange the quantitative characteristic peaks and the qualitative characteristic peaks of the compound.

In one embodiment, the method of the present invention includes: determining whether to maintain the current selection or reselect the peak ranked M in position among the top N peaks based on a re-selection parameter from the filter parameter set during the selection of the quantitative characteristic peaks, wherein the value of M is determined by the re-selection parameter.

In one embodiment, the method of the present invention includes: prior to the selection of the quantitative characteristic peaks, deleting the peaks ranked first and last in position among all the peaks of the quantitative ion pair based on a deletion parameter from the filter parameter set.

In one embodiment, the method of the present invention includes: determining whether the value of the quantity parameter is greater than or equal to 2; If the determination result is “yes,” performing a qualification judgment operation on the quantitative ion pair based on a peak ratio parameter from the filter parameter set, wherein the qualification judgment operation comprises: dividing the height of the first-ranked quantitative characteristic peak by the height of each subsequent quantitative characteristic peak to obtain one or more height ratios; determining whether each height ratio is greater than the peak ratio parameter; and if the determination result is “yes,” designating the quantitative ion pair as a qualified ion pair; otherwise, designating the quantitative ion pair as an unqualified ion pair.

The present invention provides a method for calculating the signal-to-noise ratio (S/N) of quantitative characteristic peaks, the method comprising: designating one of the quantitative characteristic peaks of the compound as a target signal; based on a S/N parameter from the filter parameter set, capturing peaks from a period before or after the position of the quantitative characteristic peak of the compound as background noise; and calculating the signal-to-noise ratio based on the intensity of the target signal and the intensity of the background noise. Preferably, the method further includes: determining whether to check the signal-to-noise ratio based on a S/N judgment parameter from the filter parameter set.

The present invention provides a method for calculating the signal-to-noise ratio (S/N) of qualitative characteristic peaks, the method comprising: designating one of the qualitative characteristic peaks of the compound as a target signal; based on a S/N parameter from the filter parameter set, capturing peaks from a period before or after the position of the qualitative characteristic peak of the compound as background noise; and calculating the signal-to-noise ratio based on the intensity of the target signal and the intensity of the background noise. Preferably, the method further includes: determining whether to check the signal-to-noise ratio based on a S/N judgment parameter from the filter parameter set.

The present invention also provides another method for obtaining characteristic peaks of a compound, the method comprising: receiving a first mass spectrometry file and a second mass spectrometry file; obtaining the characteristic peaks of a plurality of compounds from the first mass spectrometry file; reading one or more mass spectrometry data from the second mass spectrometry file, wherein each mass spectrometry data includes a set of ion pairs and all corresponding peaks belonging to a compound; and performing a characteristic peak extraction operation on the peaks read from the second mass spectrometry file based on the characteristic peaks of the compounds obtained from the first mass spectrometry file, thereby obtaining the characteristic peaks of each compound in the second mass spectrometry file.

The present invention further provides a server, which includes a characteristic peak processing program, and is configured to execute any of the methods described above based on the program code of the characteristic peak processing program.

### BRIEF DESCRIPTION OF DRAWINGS

FIG. 1 illustrates a schematic flowchart of the method of the present invention.

FIGS. 2 to 4 illustrate the mass spectra of several compounds.

FIG. 5 illustrates a schematic flowchart of another method of the present invention.

### DETAILED DESCRIPTION OF THE INVENTION

The present invention discloses a method for obtaining characteristic peaks of compounds from a mass spectrometry file, wherein the mass spectrometry file is generated by a mass spectrometer detecting a test solution. The mass spectrometry file records mass spectrometry data of one or more compounds contained in the test solution. The test solution can be prepared for various purposes. The compounds may include various pesticides (for example, 410 types) or veterinary drugs. The preparation of these solutions and the names of these pesticides can be found in the method published by the Taiwan Food and Drug Administration (TFDA) under the Ministry of Health and Welfare in 2022, titled “Method of Test for Pesticide Residues in Foods—Multiresidue Analysis (5)” (hereinafter referred to as the Published Method).

The mass spectrometry data for each compound includes a plurality of ion pairs of the compound and all corresponding peaks for each ion pair. Each peak has a height (i.e., signal intensity) and a position (i.e., retention time). Each ion pair is composed of the mass-to-charge ratio (m/z) of a precursor ion and the mass-to-charge ratio of a product ion. For example, one ion pair of Iprodione consists of a precursor ion with an m/z of 314 and a product ion with an m/z of 56, thus the ion pair is represented as 314>56. Two other ion pairs of Iprodione are represented as 314>245 and 314>271, respectively.

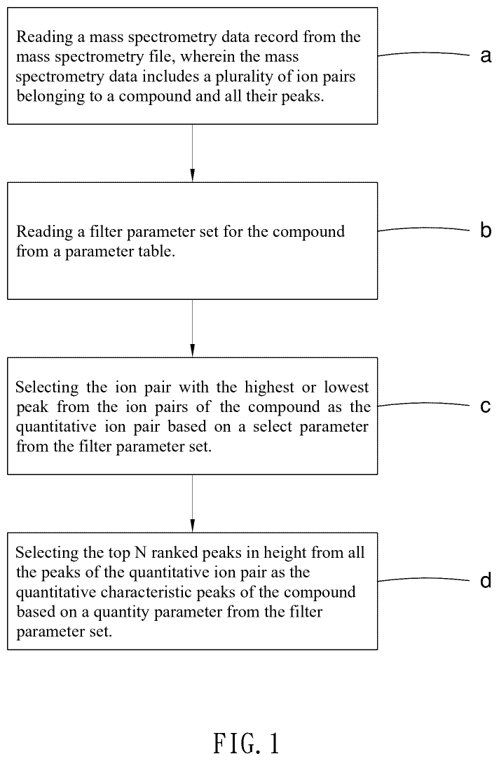

For ease of explanation, the process of obtaining the characteristic peaks of a compound is provided as an example to illustrate the steps of the method of the present invention. As shown in FIG. 1, the method of the present invention includes the following steps a˜d:
a) Reading a mass spectrometry data record from the mass spectrometry file, wherein the mass spectrometry data includes a plurality of ion pairs belonging to a compound and all their peaks;
b) Reading a filter parameter set for the compound from a parameter table; and
c) Selecting the ion pair with the highest or lowest peak from the ion pairs of the compound as the quantitative ion pair based on a select parameter from the filter parameter set; and
d) Selecting the top N ranked peaks in height from all the peaks of the quantitative ion pair as the quantitative characteristic peaks of the compound based on a quantity parameter from the filter parameter set, wherein the value of N is determined by the quantity parameter.

In step a, the mass spectrometry data that is read contains only the set of ion pairs and their corresponding peaks, but does not include the name of the compound to which they belong. If it is necessary to know the name of the compound to which they belong, the name can be obtained by querying a compound list using one of the ion pairs from the mass spectrometry data. The compound list records the names and ion pairs of each compound intended to be detected by the mass spectrometer. This compound list is usually generated along with the mass spectrometry file by the mass spectrometer, but it can also be manually created if needed.

In step b, the parameter table records the filter parameter sets for a plurality of compounds, for example, recording a filter parameter set for each compound in the compound list. For instance, if the compound list records the names and ion pairs of 410 pesticides, the parameter table should also establish the corresponding filter parameter sets for these 410 pesticides. The names of the 410 pesticides can be referenced from the Published Method.

In the aforementioned example, the mass spectrometry file contains mass spectrometry data for only one compound, so the filter parameter set for that compound is read from the parameter table to select the quantitative ion pair and quantitative characteristic peaks of the compound. However, if the mass spectrometry file contains mass spectrometry data for a plurality of compounds, the filter parameter set for those compounds must be read from the parameter table. In other words, each compound uses its own filter parameter set to select its quantitative ion pair and quantitative characteristic peaks from its respective mass spectrometry data.

In the parameter table, some compounds have the same filter parameter set, while others have different ones. Regardless, each filter parameter set includes one or more of the following parameters: a smoothing parameter, the select parameter, the quantity parameter, a position parameter, a quantitative characteristic peak re-selection parameter, a deletion parameter, and a peak ratio parameter. Preferably, it also includes a S/N parameter and a signal-to-noise ratio qualification judgment parameter.

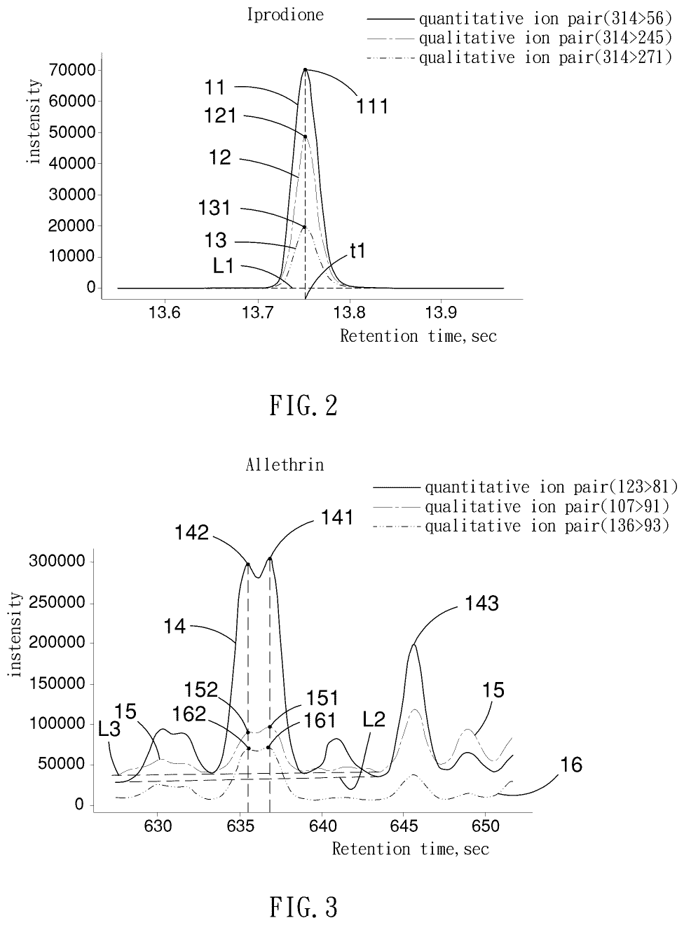

Preferably, for each peak of the ion pairs read in step b, the degree of smoothing applied to the coordinate points of the peaks can be determined based on the smoothing parameter. This helps to filter out some noise-induced peaks, making the signal curve for each ion pair, as plotted from all peaks of each ion pair (e.g., signal curves 11 to 13 in FIG. 2), as smooth as possible. For example, when the smoothing parameter is set to NULL, it indicates that no smoothing is applied. When the smoothing parameter is a numerical value, a larger value indicates a greater degree of smoothing, while a smaller value indicates a lesser degree of smoothing.

In step c, the quantitative ion pair of the compound can be selected based on the select parameter, choosing the ion pair with either the highest peak or the lowest peak. For example, if the value of the select parameter is a first value (e.g., NULL), the ion pair with the highest peak is selected as the quantitative ion pair of the compound. If the value of the select parameter is a second value (e.g., −1), the ion pair with the lowest peak is selected as the quantitative ion pair of the compound.

For example, as shown in FIG. 2, the first signal curve 11 is plotted based on all the peaks of Iprodione's first ion pair (314>56), the second signal curve 12 is plotted based on all the peaks of Iprodione's second ion pair (314>245), and the third signal curve 13 is plotted based on all the peaks of Iprodione's third ion pair (314>271). If the value of Iprodione's select parameter read from the parameter table is the first value, Iprodione's first ion pair (314>56) is selected as the quantitative ion pair because, among the three curves 11 to 13, the first ion pair (314>56) has the highest peak 111. The other two ion pairs (314>245 and 314>271) are then designated as the two qualitative ion pairs (qualitative ion pairs) of Iprodione. Accordingly, the first signal curve 11 represents the quantitative ion pair signal curve, while the second and third signal curves 12 and 13 represent the qualitative ion pair signal curves. In this way, the selection of one quantitative ion pair (314>56) and two qualitative ion pairs (314>245 and 314>271) for Iprodione is completed.

Similarly, as shown in FIG. 3, among the three signal curves 14 to 16 plotted based on all the peaks of Allethrin's three ion pairs, the first ion pair (123>81) has the highest peak 141. Therefore, this ion pair (123>81) is selected as Allethrin's quantitative ion pair, while the other two ion pairs (107>91 and 136>93) are designated as Allethrin's two qualitative ion pairs. Accordingly, signal curve 14 represents the quantitative ion pair signal curve, while signal curves 15 and 16 represent the qualitative ion pair signal curves. In this way, the selection of one quantitative ion pair (123>81) and two qualitative ion pairs (107>91 and 136>93) for Allethrin is completed.

In step c, if the value of Cypermethrin's quantitative ion pair selection parameter read from the parameter table is the second value, Cypermethrin's third ion pair (181>152.1) is temporarily selected as the quantitative ion pair, and the other two ion pairs (163>91 and 63>127) are temporarily selected as the two qualitative ion pairs. This is because, as shown in FIG. 4, among the three signal curves 17 to 19 plotted based on all the peaks of Cypermethrin's three ion pairs, the third ion pair (181>152.1) has the lowest peak 191. Once Cypermethrin's quantitative characteristic peaks 191 to 194 (temporary) and qualitative characteristic peaks 181 to 184 (temporary) are identified in step d, a swapping operation is performed to interchange Cypermethrin's quantitative characteristic peaks 191 to 194 (temporary) with the qualitative characteristic peaks 181 to 184 (temporary), thereby obtaining Cypermethrin's true quantitative characteristic peaks 181 to 184 and true qualitative characteristic peaks 191 to 194. Consequently, the previously temporary quantitative ion pair (181>152.1) is re-designated as the qualitative ion pair, and the previously temporary qualitative ion pair (163>127) is re-designated as the quantitative ion pair. Thus, in FIG. 4, 163>127 is Cypermethrin's quantitative ion pair, 181>152.1 is Cypermethrin's qualitative ion pair, and the quantitative ion pair signal curve and qualitative ion pair signal curve correspond to FIG. 18 and FIG. 19, respectively. Although signal curve 17 has the highest peak 171, it only represents one of Cypermethrin's ion pairs (163>91) and is neither the quantitative ion pair signal curve nor the qualitative ion pair signal curve.

In step d, the number of quantitative characteristic peaks N for the compound can be determined based on the quantity parameter. For example, if the value of the quantity parameter is NULL or 1, the value of N is 1. In this case, the peak ranked first in height among all the peaks of the compound's quantitative ion pair is selected as the only quantitative characteristic peak of the compound. If the value of the quantity parameter is 2, the value of N is 2, and the top 2 peaks ranked by height (i.e., the 1st and 2nd peaks) among all the peaks of the compound's quantitative ion pair are selected as the two quantitative characteristic peaks of the compound. If the value of the quantity parameter is 3, the value of N is 3, and the top 3 peaks ranked by height (i.e., the 1st to 3rd peaks) among all the peaks of the compound's quantitative ion pair are selected as the three quantitative characteristic peaks of the compound, and so on.

For example, if the value of Iprodione's quantity parameter read from the parameter table is NULL, it indicates that N=1. In this case, as shown in FIG. 2, the highest peak 111, which is ranked first among all the peaks of Iprodione's quantitative ion pair (314>56), is selected as the only quantitative characteristic peak of Iprodione's quantitative ion pair (314>56). If the value of Allethrin's quantity parameter read from the parameter table is 2, it indicates that N=2. In this case, as shown in FIG. 3, although the heights of peaks 141 to 143 from Allethrin's quantitative ion pair (123>81) are all significant, only the top 2 peaks 141 and 142, ranked by height, are selected as the two quantitative characteristic peaks of Allethrin's quantitative ion pair (123>81) because the quantity parameter value is 2. If the value of Cypermethrin's quantity parameter read from the parameter table is 4, it indicates that N=4. In this case, as shown in FIG. 4, the top 4 peaks 191 to 194, ranked by height from Cypermethrin's quantitative ion pair (181>152.1, temporarily), are temporarily selected as Cypermethrin's four quantitative characteristic peaks.

After filtering out the compound's quantitative ion pair and quantitative characteristic peaks, the qualitative characteristic peaks of the compound can be identified based on their positions. Specifically, an allowable position range can be defined for each of the quantitative characteristic peaks based on the position parameter, and peaks whose positions fall within this allowable range, from ion pairs other than the quantitative ion pair (i.e., qualitative ion pairs), are selected as the compound's qualitative characteristic peaks. For example, if the position parameter is NULL or 1, the allowable position range for each quantitative characteristic peak is within 1 second before and after its position. If the position parameter is 1.5, the range extends to 1.5 seconds before and after the position of each quantitative characteristic peak. If the position parameter is 2, the allowable range is 2 seconds before and after, and so on.

For example, in FIG. 2, the position of Iprodione's quantitative characteristic peak (i.e., the highest peak 111) is t1. If the value of Iprodione's position parameter read from the parameter table is 1, the allowable position range for peak 111 is between t1−1 and t1+1. Next, by tracing down from the quantitative characteristic peak 111 along a dashed line passing through position t1 (a virtual line that does not exist in the mass spectrum), the peak 121 at position t1, as well as the peak 131 near position t1, can be found. Since both of these peaks 121 and 131 fall within the allowable position range of quantitative characteristic peak 111, they are selected as Iprodione's two qualitative characteristic peaks, 121 and 131. Thus, a total of three characteristic peaks are obtained for Iprodione: one quantitative characteristic peak 111 and two qualitative characteristic peaks 121 and 131.

Similarly, Allethrin's qualitative characteristic peaks can be identified based on the positions of its two quantitative characteristic peaks, 141 and 142. As shown in FIG. 3, peaks 151 and 152 correspond to the positions of the two quantitative characteristic peaks 141 and 142, so they are designated as the two qualitative characteristic peaks for one of Allethrin's qualitative ion pairs (107>91). Additionally, peaks 161 and 162 are still within the allowable position range of quantitative characteristic peaks 141 and 142, so they are designated as the two qualitative characteristic peaks for Allethrin's other qualitative ion pair (136>93). Thus, a total of six characteristic peaks are obtained for Allethrin: two quantitative characteristic peaks 141 and 142, and four qualitative characteristic peaks 151, 152, 161, and 162.

Similarly, the qualitative characteristic peaks (temporary) of Cypermethrin can be identified based on the positions of its four quantitative characteristic peaks 191 to 194 (temporary). As shown in FIG. 4, by tracing up along the dashed lines through the quantitative characteristic peaks 191 to 194, peaks 181 to 184 can be found on one of Cypermethrin's qualitative ion pair curves (163>127), and they are temporarily designated as the four qualitative characteristic peaks of this qualitative ion pair. Then, the aforementioned swapping operation is performed to obtain eight characteristic peaks for Cypermethrin, namely, the four quantitative characteristic peaks 181 to 184 and the four qualitative characteristic peaks 191 to 194.

As described above, the method of the present invention can indeed obtain characteristic peaks of one or more compounds from any mass spectrometry file. The characteristic peaks of each compound include the quantitative characteristic peaks of a quantitative ion pair, and preferably, one or more qualitative characteristic peaks from qualitative ion pairs. The number of characteristic peaks for each quantitative or qualitative ion pair may be one, two, or more.

In step d, for some compounds, not all the peaks identified based on the quantity parameter (i.e., the top N peaks by height) may be suitable to be designated as quantitative characteristic peaks. To address this, the method of the present invention may further include: determining, based on the quantitative characteristic peak re-selection parameter, whether to retain all the top N peaks as the compound's quantitative characteristic peaks, or to reselect the peak ranked at position M among the top N peaks as the compound's quantitative characteristic peak, where M is determined by the re-selection parameter. For example, when the value of the re-selection parameter is NULL, M is also NULL, and all the top N peaks are designated as the compound's quantitative characteristic peaks. However, if the re-selection parameter is not NULL, the peak ranked at position M among the top N peaks is designated as the compound's quantitative characteristic peak. For instance, if the value of the re-selection parameter is 1, M equals 1, and the peak ranked first among the top N peaks is designated as the compound's sole quantitative characteristic peak. If the value of the re-selection parameter is 2, M equals 2, and the peak ranked second among the top N peaks is designated as the sole quantitative characteristic peak, and so on. Assuming the value of Cypermethrin's re-selection parameter read from the parameter table is 2, as shown in FIG. 4, the second peak from the left among the top four peaks 191 to 194 is peak 192, so only peak 192 is temporarily designated as Cypermethrin's sole quantitative characteristic peak.

Considering that the highest peak of the quantitative ion pair of some compounds may coincidentally be the first or last peak in terms of position, this means that the starting or ending point of the quantitative ion pair curve for these compounds could be the highest peak. If this is the case, during step d, the first or last peak may mistakenly be identified as one of the compound's quantitative characteristic peaks, which would be incorrect. To avoid this error, the method of the present invention may further include: deleting the first and last peaks of all the peaks in the compound's quantitative ion pair based on the deletion parameter, and then proceeding with step d. For example, when the deletion parameter has a first value (e.g., NULL), the first and last peaks of the compound's quantitative ion pair are deleted, and then step d is continued. However, when the deletion parameter has a second value (e.g., −1), it indicates that no peaks should be deleted. Additionally, if the quantity parameter is ≥2, no peaks need to be deleted (i.e., the deletion parameter can be ignored).

In step c, considering that some compounds' quantitative ion pairs, after being selected, may require further evaluation to determine if they are qualified, the method of the present invention may also include: determining whether the value of the compound's quantity parameter is greater than or equal to 2. If the result is “yes,” a qualification judgment operation is performed on the compound's quantitative ion pair based on the compound's peak ratio parameter. This qualification judgment operation includes: dividing the height of the first-ranked quantitative characteristic peak by the height of each subsequent quantitative characteristic peak to obtain one or more height ratios; determining whether each height ratio is greater than the peak ratio parameter; and if the result is “yes,” designating the quantitative ion pair as a qualified ion pair, otherwise, designating it as an unqualified ion pair.

In the method of the present invention, when selecting the quantitative ion pair and quantitative characteristic peaks for each compound, each compound uses the rules defined by its own filter parameter set, thereby significantly improving the accuracy of the obtained characteristic peaks. Furthermore, the parameter table is specifically established for the mass spectrometer that generated the mass spectrometry file. In other words, separate parameter tables are created for different brands of mass spectrometers, or even for the same brand and model used by different users, fully integrating instrument characteristics and user habits to further improve the accuracy of the obtained characteristic peaks.

The invention also discloses a method for calculating the signal-to-noise ratio (S/N), comprising:
Capturing peaks from a period before or after the position of the compound's characteristic peak (e.g., 10 seconds) based on a S/N parameter in the compound's filter parameter set, treating these peaks as background noise, and treating the compound's characteristic peak as the target signal, where the characteristic peak can be either a quantitative or qualitative characteristic peak; and
Calculating the signal-to-noise ratio (S/N) for the characteristic peak based on the intensity of the target signal and the intensity of the background noise.

Preferably, the method for calculating the signal-to-noise ratio may further include: determining, based on the signal-to-noise ratio qualification judgment parameter in the compound's filter parameter set, whether to check the signal-to-noise ratio of the characteristic peak. For any compound, the signal-to-noise ratio of a quantitative characteristic peak typically must be greater than or equal to 10 to be considered qualified; otherwise, it is considered unqualified. For any compound, the signal-to-noise ratio of a qualitative characteristic peak typically must be greater than or equal to 2 to be considered qualified; otherwise, it is considered unqualified.

The method of the present invention uses a parameter table to filter a mass spectrometry file in order to obtain the characteristic peaks of compounds. The following discloses another method of the present invention, which utilizes peak matching to obtain the characteristic peaks of compounds.

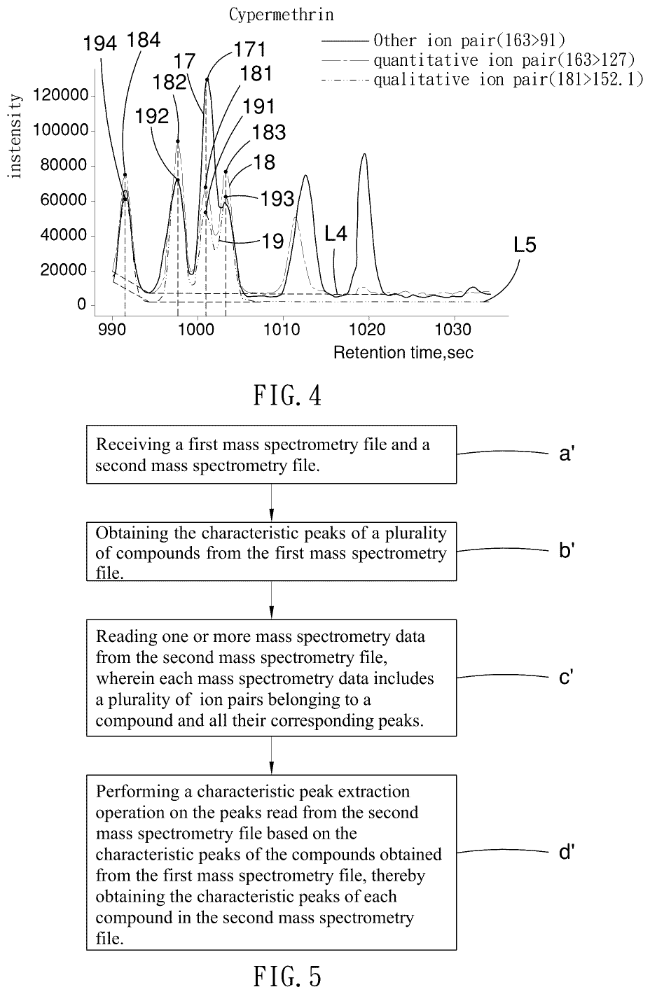

Another method for obtaining the characteristic peaks of compounds from a mass spectrometry file, as shown in FIG. 5, comprises the following steps a′˜d′:
a′) Receiving a first mass spectrometry file and a second mass spectrometry file;
b′) Obtaining the characteristic peaks of a plurality of compounds from the first mass spectrometry file;
c′) Reading one or more mass spectrometry data from the second mass spectrometry file, wherein each mass spectrometry data includes a plurality of ion pairs belonging to a compound and all their corresponding peaks; and
d′) Performing a characteristic peak extraction operation on the peaks read from the second mass spectrometry file based on the characteristic peaks of the compounds obtained from the first mass spectrometry file, thereby obtaining the characteristic peaks of each compound in the second mass spectrometry file.

In step a′, it is preferable that the first mass spectrometry file and the second mass spectrometry file are generated by the same mass spectrometer, but they can also be generated by different mass spectrometers. The first mass spectrometry file records one or more mass spectrometry data generated by a mass spectrometer detecting a first test solution. The second mass spectrometry file records one or more mass spectrometry data generated by a mass spectrometer detecting a second test solution. Each mass spectrometry data includes a plurality of ion pairs belonging to a compound and all corresponding peaks for each ion pair.

In one embodiment, the first test solution can be a standard solution, and the second test solution can be a sample solution. In this case, the first and second mass spectrometry files generated by the mass spectrometer are a standard mass spectrometry file and a sample mass spectrometry file, respectively.

The standard solution contains a matrix and a compound standard solution with a predetermined concentration (e.g., 50 ppb). The matrix is a sample that does not contain any compounds, which can be derived from crops or foods that are free of pesticides and other chemicals. The compound standard solution contains one or more compound standards, such as a pesticide standard solution containing a plurality of pesticide standards like abamectin, acephate, and others. If the standard solution contains 216 pesticide standards (but is not limited to this number), the mass spectrometry data for these 216 pesticide standards will be recorded in the standard mass spectrometry file (i.e., the first mass spectrometry file) generated by the mass spectrometer when analyzing the standard solution. The sample solution contains a test sample, which can be derived from crops or foods. If the sample solution contains several compounds (e.g., pesticides or veterinary drugs), the mass spectrometry data for these compounds will be recorded in the sample mass spectrometry file (i.e., the second mass spectrometry file) generated by the mass spectrometer when analyzing the sample solution.

In step b′, the method for obtaining the characteristic peaks of each compound is preferably the method described in FIG. 1 of the present invention (but not limited to this), and will not be repeated here. When the first mass spectrometry file is the standard mass spectrometry file, completing step b′ will result in obtaining the characteristic peaks of the 216 pesticide standards mentioned earlier, which serve as the reference for the subsequent characteristic peak extraction operation.

In step c′, the process of reading the second mass spectrometry file is generally the same as step b in FIG. 1, and will not be repeated here.

In step d′, the characteristic peak extraction operation is performed by identifying peaks from the second mass spectrometry file that have the same or similar positions as the characteristic peaks (e.g., quantitative characteristic peaks) of each compound, and designating the identified peaks as characteristic peaks.

The term “same or similar position” refers to a deviation within a certain time range (e.g., ±3 seconds). For example, when the position of one of Allethrin's quantitative characteristic peaks from the first mass spectrometry file (e.g., the standard mass spectrometry file) is at time t, three peaks at position t from Allethrin's three ion pairs in the second mass spectrometry file (e.g., the sample mass spectrometry file) are identified. The peak with the highest intensity among these three is designated as one of Allethrin's quantitative characteristic peaks for the quantitative ion pair, and the other two peaks are designated as one of the qualitative characteristic peaks for each of Allethrin's two qualitative ion pairs. However, if no such peaks are found, the search is extended to the time range t−3 to t+3, and the identified peaks are then designated as the aforementioned quantitative and qualitative characteristic peaks. Other quantitative and qualitative characteristic peaks of Allethrin from the second mass spectrometry file can be found using this method. Similarly, the quantitative and qualitative characteristic peaks of other compounds from the second mass spectrometry file can also be identified using this method.

The quantitative or qualitative characteristic peaks of each compound from the second mass spectrometry file can be obtained using the above steps a′ to c′. However, the qualitative characteristic peaks of each compound from the second mass spectrometry file can also be obtained by using the positions of the already identified quantitative characteristic peaks.

As described above, using characteristic peaks obtained from one mass spectrometry file to match peaks in another mass spectrometry file allows for quickly and accurately identifying the characteristic peaks—whether quantitative or qualitative—from the compounds in the other mass spectrometry file.

Using the method of the present invention as shown in FIG. 5, the characteristic peaks of the 216 pesticide standards can be obtained from the standard mass spectrometry file, and these characteristic peaks can be used as matching references to obtain the characteristic peaks of one or more pesticides from the sample mass spectrometry file. Similarly, the characteristic peaks of various pesticide standards obtained from the standard mass spectrometry file can be used to obtain the characteristic peaks of one or more compounds from other mass spectrometry files.

After obtaining the characteristic peaks of compounds using the method shown in FIG. 1 or FIG. 5, the total area and signal-to-noise ratio (S/N) of the quantitative characteristic peaks, the total area and S/N of the qualitative characteristic peaks, and the area ratio for each compound can be calculated based on the obtained characteristic peaks. The total area of both the quantitative and qualitative characteristic peaks can be calculated using the composite trapezoidal rule, but is not limited to this. The S/N of the quantitative characteristic peaks is the ratio of the signal intensity of the quantitative characteristic peaks to the nearby background noise. Similarly, the S/N of the qualitative characteristic peaks is the ratio of the signal intensity of the qualitative characteristic peaks to the nearby background noise. The area ratio is defined as (total area of the qualitative characteristic peaks)/(total area of the quantitative characteristic peaks).

When calculating the total area of a characteristic peak, the method of the present invention first establishes a baseline for the characteristic peak (see baselines L1 to L5 in FIGS. 2 to 4), and then uses the composite trapezoidal rule to calculate the total area of the characteristic peak. The baseline is the line connecting the lowest points on either side of the characteristic peak. In this embodiment, the method of the present invention establishes one baseline for each quantitative and qualitative characteristic peak of each compound. As shown in FIG. 2, since the lowest points of Iprodione's quantitative ion pair curve 11 and qualitative ion pair curve 12 overlap, there is only one baseline, L1. As shown in FIG. 3, there is a baseline L2 for Allethrin's quantitative ion pair curve 14 and a baseline L3 for the qualitative ion pair curve 15. As shown in FIG. 4, there is a baseline L4 for Cypermethrin's quantitative ion pair curve 18 and a baseline L5 for the qualitative ion pair curve 19.

The present invention also discloses a server, which is coupled to a client computer, and the mass spectrometer is coupled to the client computer. The mass spectrometer can be a liquid chromatography-tandem mass spectrometer (LC/MS/MS), a gas chromatography-tandem mass spectrometer (GC/MS/MS), or other types of mass spectrometers. The mass spectrometry file produced by the mass spectrometer is created on the client computer and uploaded to the server via the network. The server typically consists of one or more server-grade computers and one or more storage devices, but is not limited to this configuration.

The server is installed with a characteristic peak processing program and can execute any of the methods described in this invention based on the program code of the characteristic peak processing program. Preferably, the server is also installed with a conversion program that converts the file format of the mass spectrometry file into the format required by the characteristic peak processing program, such as commonly known standard mass spectrometry file formats: mzData, mzXML, or mzML. In one embodiment, the file format required by the characteristic peak processing program is mzML. In another embodiment, if the original file format of the mass spectrometry file produced by the mass spectrometer is already in the format required by the characteristic peak processing program, the conversion program is not necessary, and thus, no file format conversion is needed.

In summary, the present invention utilizes the server to execute the aforementioned methods, enabling the automatic and accurate acquisition of one or more characteristic peaks of compounds from a mass spectrometry file, which not only improves the overall detection speed but also prevents human errors and addresses the issue of labor shortages.

1. A method for obtaining characteristic peaks of a compound, comprising:
reading a mass spectrometry data from a mass spectrometry file, wherein the mass spectrometry data includes a plurality of ion pairs belonging to the compound and all their corresponding peaks;
reading a filter parameter set for the compound from a parameter table;
selecting an ion pair with the highest or lowest peak from the ion pairs as a quantitative ion pair of the compound based on a select parameter in the filter parameter set; and
selecting the top N peaks by height from all the peaks of the quantitative ion pair as quantitative characteristic peaks of the compound based on a quantity parameter in the filter parameter set, where the value of N is determined by the quantity parameter.

2. The method as recited in claim 1, wherein the parameter table is established for a mass spectrometer that generated the mass spectrometry file.

3. The method as recited in claim 2, further comprising:
before reading the filter parameter set, obtaining the name of the compound by querying a compound list based on an ion pair from the mass spectrometry data, wherein the compound list records the name and ion pairs of each compound intended to be detected by the mass spectrometer, and the parameter table records the name and the filter parameter set of each compound in the compound list.

4. The method as recited in claim 1, further comprising:
based on the position of the quantitative characteristic peaks of the compound, identifying peaks with the same or similar positions from all peaks of ion pairs other than the quantitative ion pair as qualitative characteristic peaks of the compound.

5. The method as recited in claim 1, further comprising:
defining an allowable position range for each of the quantitative characteristic peaks based on a position parameter from the filter parameter set; and
selecting peaks from all the peaks of ion pairs other than the quantitative ion pair, where the position of the peaks falls within the allowable position range, as qualitative characteristic peaks of the compound.

6. The method as recited in claim 5, wherein, after obtaining the qualitative characteristic peaks of the compound, if the quantitative ion pair is selected from the ion pair with the lowest peak, a swapping operation should be performed to interchange the quantitative characteristic peaks and the qualitative characteristic peaks of the compound.

7. The method as recited in claim 1, further comprising:
during the selection of the quantitative characteristic peaks, determining whether to maintain the current selection or reselect the peak ranked M in position among the top N peaks as quantitative characteristic peaks of the compound based on a re-selection parameter from the filter parameter set, wherein the value of M is determined by the re-selection parameter.

8. The method as recited in claim 1, further comprising:
prior to the selection of the quantitative characteristic peaks, deleting the peaks ranked first and last in position among all the peaks of the quantitative ion pair based on a deletion parameter from the filter parameter set.

9. The method as recited in claim 1, further comprising:
determining whether the value of the quantity parameter is greater than or equal to 2;
if the determination result is “yes,” performing a qualification judgment operation on the quantitative ion pair based on a peak ratio parameter from the filter parameter set, wherein the qualification judgment operation comprises:
dividing the height of the first-ranked quantitative characteristic peak by the height of each subsequent quantitative characteristic peak to obtain one or more height ratios;
determining whether each height ratio is greater than the peak ratio parameter; and
if the determination result is “yes,” designating the quantitative ion pair as a qualified ion pair; otherwise, designating the quantitative ion pair as an unqualified ion pair.

10. A method for calculating the signal-to-noise ratio (S/N) of quantitative characteristic peaks, comprising:
obtaining the quantitative characteristic peaks of the compound using the method described in claim 1;
designating one of the quantitative characteristic peaks of the compound as a target signal;
based on a S/N parameter from the filter parameter set, capturing peaks from a period before or after the position of the quantitative characteristic peak of the compound as a background noise; and
calculating a signal-to-noise ratio based on the intensity of the target signal and the intensity of the background noise.

11. The method as recited in claim 10, further comprising:
determining whether to check the signal-to-noise ratio based on a S/N judgment parameter from the filter parameter set.

12. A method for calculating the signal-to-noise ratio (S/N) of qualitative characteristic peaks, comprising:
obtaining the qualitative characteristic peaks of the compound using the method described in claim 4;
designating one of the qualitative characteristic peaks of the compound as a target signal;
based on a S/N parameter from the filter parameter set, capturing peaks from a period before or after the position of the qualitative characteristic peak of the compound as background noise; and
calculating a signal-to-noise ratio based on the intensity of the target signal and the intensity of the background noise.

13. The method as recited in claim 12, further comprising:
determining whether to check the signal-to-noise ratio of
the qualitative characteristic peak based on a S/N judgment parameter from the filter parameter set.

14. A method for obtaining characteristic peaks of compounds, comprising:
receiving a first mass spectrometry file and a second mass spectrometry file;
obtaining the characteristic peaks of a plurality of compounds from the first mass spectrometry file;
reading one or more mass spectrometry data from the second mass spectrometry file, wherein each mass spectrometry data includes a set of ion pairs and all corresponding peaks belonging to a compound; and
performing a characteristic peak extraction operation on the peaks read from the second mass spectrometry file based on the characteristic peaks of the compounds obtained from the first mass spectrometry file, thereby obtaining the characteristic peaks of each compound in the second mass spectrometry file.

15. A server configured to perform the method as recited in claim 1.
